(19) United States
(12) Patent Application Publication
(10) Pub. No.: US 2010/0216146 A1
(43) Pub. Date: Aug. 26, 2010

(54) Methods and Kits for Hybridizing Multiple PNA Probe Panels to Nucleic Acid Samples

(75) Inventors: Brett Williams, Eugene, OR (US); Jack Johansen, Concord, MA (US); Jens J. Hyldig-Nielsen, Moss Beach, CA (US)

Correspondence Address: LIFE TECHNOLOGIES CORPORATION;C/O INTELLEVATE, P.O. BOX 52050, MINNEAPOLIS, MN, 55402, US

(73) Assignee: APPLIED BIOSYSTEMS, LLC, Carlsbad, CA (US)

(21) Appl. No.: 12/693,235

(22) Filed: Jan. 25, 2010

Related U.S. Application Data
(63) Continuation of application No. 10/954,802, filed on Sep. 29, 2004, now abandoned.
(60) Provisional application No. 60/507,787, filed on Sep. 30, 2003.

(51) Int. Cl. C12Q 1/68

(52) U.S. Cl. 435/6

(57) ABSTRACT

Described herein are methods and kits that employ multiple probe sets in combination with sequential steps of hybridization analysis for multiplex analysis and/or detection of nucleic acids having one or more distinguishable target sequences.

### CROSS-REFERENCES TO RELATED APPLICATIONS

This application claims benefit under 35 U.S.C. §119(e) to application Ser. No. 60/507,787, entitled “Methods and Kits for Hybridizing Multiple Probe Panels to Nucleic Acid Samples,” filed Sep. 30, 2003, the disclosure of which is incorporated herein by reference in its entirety.

### FIELD

The present disclosure relates generally to the field of nucleic acid analysis.

### INTRODUCTION

Sequence specific nucleic acid hybridization is fundamental to molecular biological processes. Probe-based assays that exploit sequence-specific hybridization can be used in many applications such as for detecting, analyzing, quantifying and/or locating nucleic acids. For example, probe-base hybridization can be employed to quantify gene expression levels, to detect single nucleotide polymorphisms (SNP) and/or other genetic mutations, as well as to type, map and/or fingerprint genes or to diagnose chromosome abnormalities. Probe-base hybridization can be applied to pathogen identification as well as to numerous other applications.

Oftentimes, such probe-based hybridization assays are carried out in a multiplex fashion with probes bearing different, distinguishable labels thereby permitting a multiplicity of results to be obtained in a single assay reaction. While such multiplex assays can be powerful, the number of different sequences that can be assessed in a single assay reaction can be limited by several factors, including, for example, the number of different, distinguishable labels available, and the availability of detection equipment capable of detecting the signals produced by the different, distinguishable labels. The versatility of multiplex assays might be improved by the ability to analyze the same sample more than once.

### BRIEF DESCRIPTION OF THE DRAWINGS

The skilled artisan will understand that the drawings, described below, are for illustration purposes only. The drawings are not intended to limit the scope of the present teachings in any way.

FIG. 1 illustrates the function of a signal probe and two possible competitor probe embodiments in the analysis of a nucleic acid molecule;

FIG. 2A illustrates an embodiment that uses two sets of signal probes and one set of competitor probes, wherein the competitor probes are at least partially identical to the signal probes;

FIG. 2B illustrates an embodiment that uses two sets of signal probes and one set of competitor probes, wherein the competitor probes are at least partially complementary to the signal probes;

FIG. 2C illustrates an embodiment in which a nucleic acid is simultaneously contacted with a second set of signal probes and a set of competitor probes;

FIG. 2D illustrates the step of contacting a nucleic acid with a set of signal probes comprising 4 subsets;

FIG. 2E illustrates an embodiment for analyzing the sample with multiple probe sets in which the competitor probes includes sequences that are substantially complementary and substantially identical to the signal probes; and

FIG. 3A-C are images of actual in situ detection of human chromosomes using two signal probe sets and a competitor probe set wherein the competitor probe set comprises probes that are substantially identical to the signal probes of the first signal probe set.

### DESCRIPTION

### Abbreviations and Conventions

The abbreviations used throughout the specification and in the FIGS. to refer to nucleic acid sequences and probes comprising specific nucleobase sequences are the conventional one-letter abbreviations. Capital letters represent nucleotide sequences (e.g., RNA and DNA sequences) and lower case letters represent nucleotide mimic sequences (e.g., PNA sequences). Thus, when included in a poly or oligonucleotide, the naturally occurring encoding nucleobases are abbreviated as follows: adenine (A), guanine (G), cytosine (C), thymine (T) and uracil (U). When included in a poly or oligonucleotide mimic, such as a PNA, the naturally occurring encoding nucleobases are abbreviated as follows: adenine (a), guanine (g), cytosine (c), thymine (t) and uracil (u). “Nucleobase sequence” or “sequence” are used interchangeably.

Also, unless specified otherwise, poly or oligonucleotide sequences that are represented as a series of one-letter abbreviations are presented in the 3′→5′ direction, in accordance with common convention. PNAs are presented in the amino-to-carboxy direction, in accordance with common convention. For the purposes of distinguishing parallel from anti-parallel hybridization orientation, it is understood that the 5′ terminus of an oligonucleotide corresponds to the amino terminus of a PNA and the 3′ terminus of an oligonucleotide corresponds to the carboxy terminus of a PNA.

### DEFINITIONS

As used throughout the specification and claims, the following terms are intended to have the definitions delineated below. Terms defined in the singular also include the plural and vice versa.

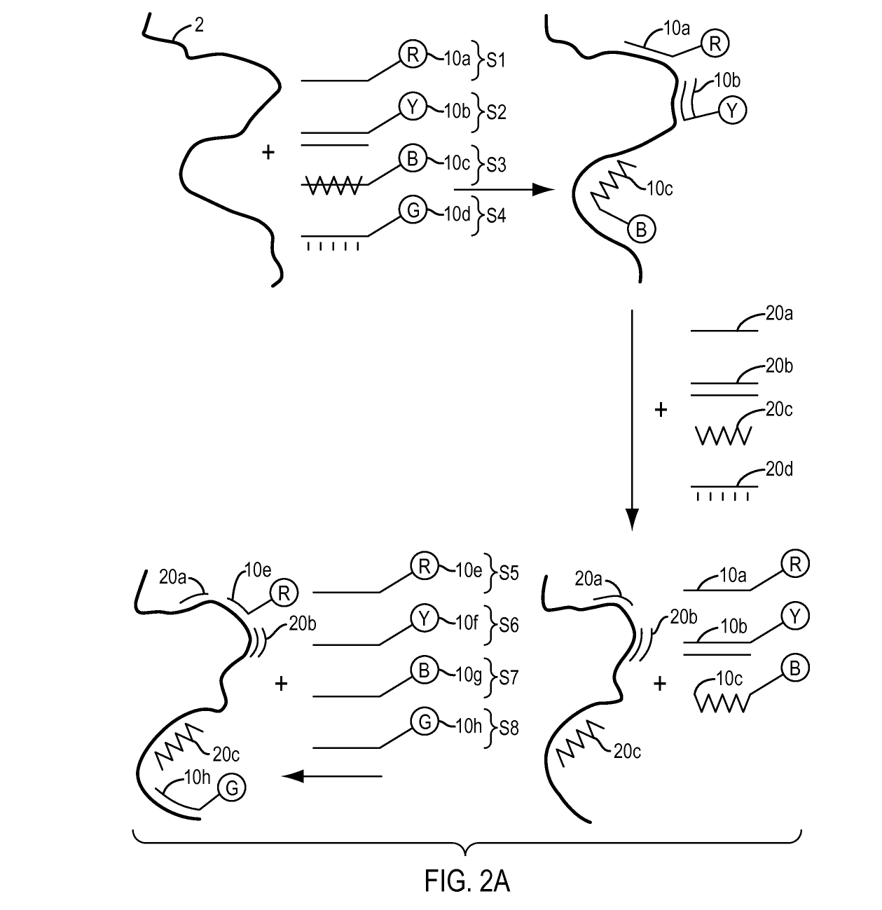

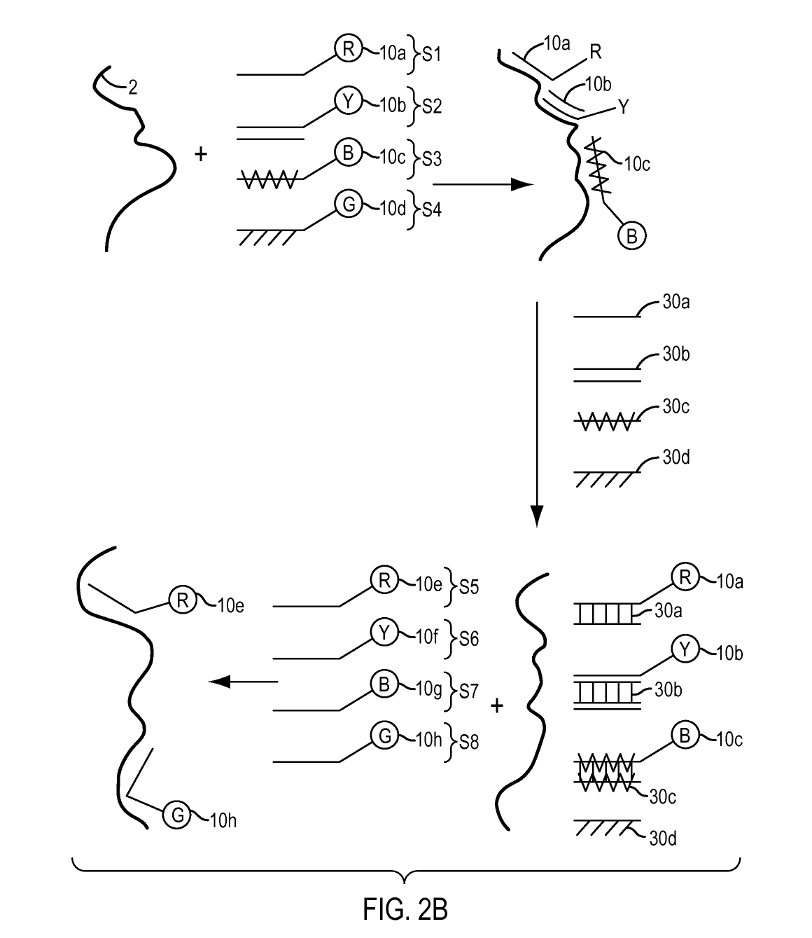

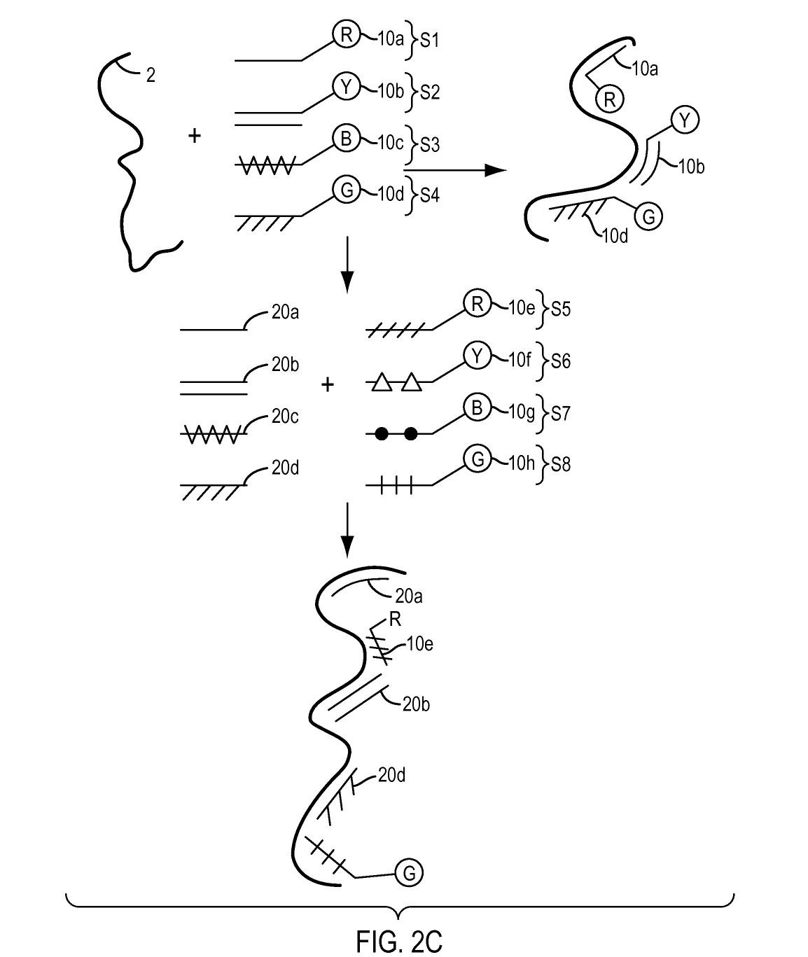

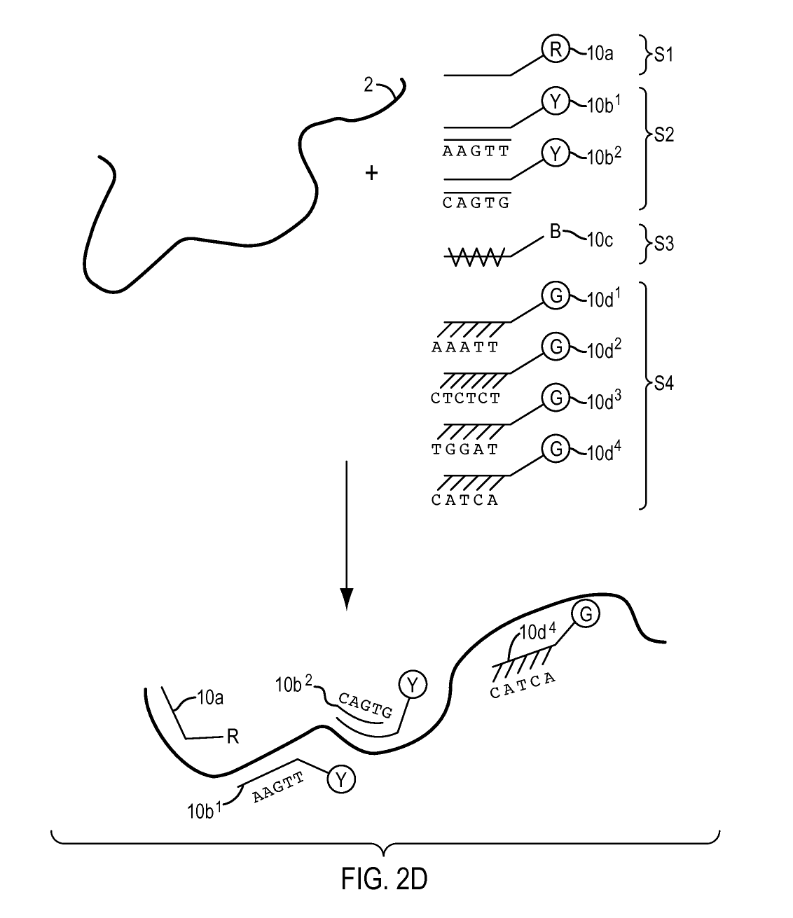

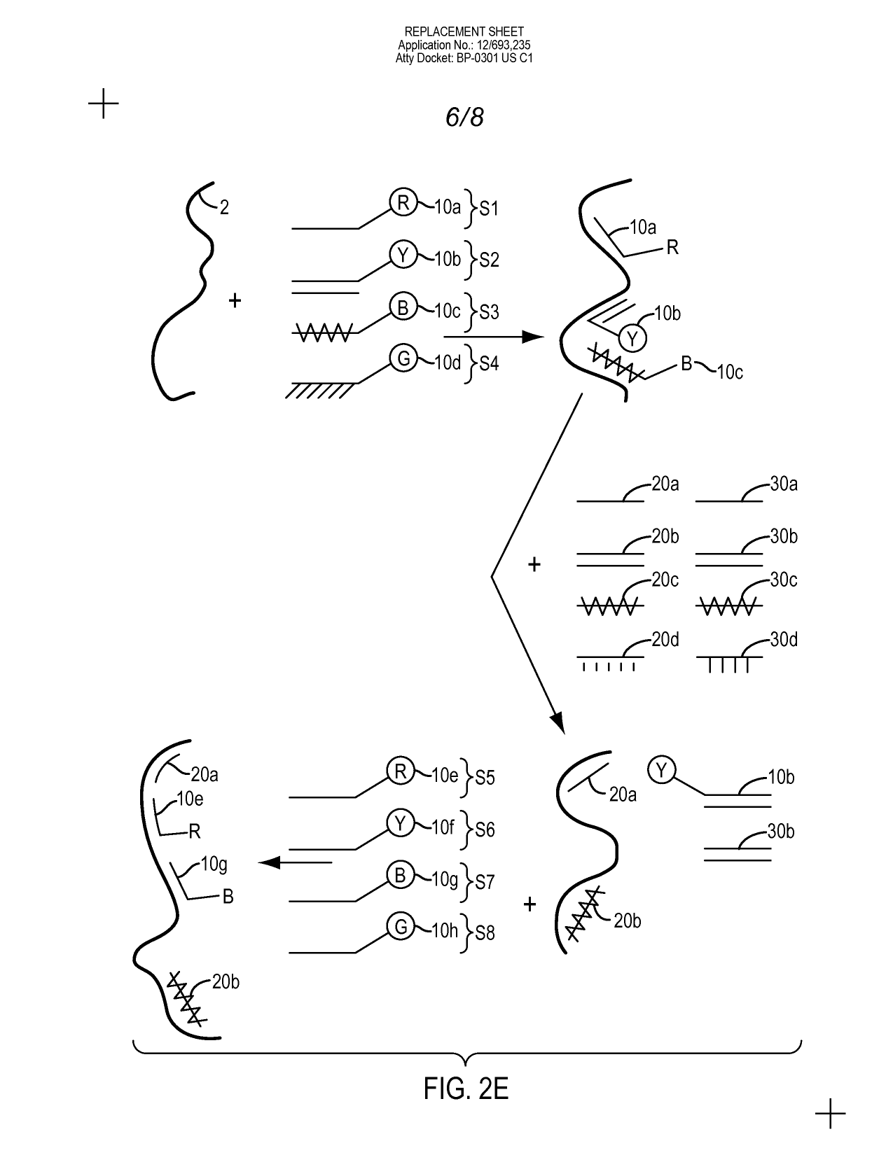

“Nucleobase” means those naturally occurring and those synthetic nitrogenous, aromatic moieties commonly found in the nucleic acid arts. Examples of nucleobases include purines and pyrimidines, genetically encoding nucleobases, analogs of genetically encoding nucleobases, and purely synthetic nucleobases. Specific examples of genetically encoding nucleobases include adenine, cytosine, guanine, thymine, and uracil. Specific examples of analogs of genetically encoding nucleobases and synthetic nucleobases include 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). 5-propynyl-uracil, 2-thio-5-propynyl-uracil. Other non-limiting examples of suitable nucleobases include those nucleobases illustrated in FIGS. 2(A) and 2(B) of U.S. Pat. No. 6,357,163, incorporated herein by reference in its entirety.

“Nucleoside” refers to a nucleobase linked to a pentose sugar. Pentose sugars include ribose, 2′-deoxyribose, 3′-deoxyribose, and 2′,3′-dideoxyribose.

“Nucleoside analog” refers to a nucleobase linked to a sugar, other than a pentose sugar. For example, a nucleobase linked to hexose.

“Nucleotide” refers to compound comprising a nucleobase, a pentose sugar and a phosphate. Thus, as used herein a nucleotide refers to a phosphate ester of a nucleoside, e.g., a triphosphate.

“Nucleobase Polymer or Oligomer” refers to two or more nucleobases that are connected by linkages that permit the resultant nucleobase polymer or oligomer to hybridize to a polynucleotide having at least a partially complementary nucleobase sequence. Nucleobase polymers or oligomers include, but are not limited to, poly- and oligonucleotides (e.g., DNA and RNA polymers and oligomers), poly- and oligonucleotide analogs and poly- and oligonucleotide mimics, such as polyimide nucleic acids or peptide nucleic acids. Polyamide nucleic acids and peptide nucleic acids are two different phrases used in the literature to describe the same molecule, abbreviated herein as PNA. Nucleobase polymers or oligomers can vary in size from a few nucleobases, for example, from 2 to 40 nucleobases, to several hundred nucleobases, to several thousand nucleobases, or more.

“Polynucleotides or Oligonucleotides” refer to nucleobase polymers or oligomers in which the nucleobases are linked by sugar phosphate linkages (sugar-phosphate backbone). Exemplary poly- and oligonucleotides include polymers of 2′-deoxyribonucleotides (DNA) and polymers of ribonucleotides (RNA). A polynucleotide may be composed entirely of ribonucleotides, entirely of 2′-deoxyribonucleotides or combinations thereof.

“Polynucleotide or Oligonucleotide Analog” refers to nucleobase polymers or oligomers in which the nucleobases are linked by a phosphate backbone comprising one or more sugar analogs or phosphate analogs. Typical oligonucleotide or polynucleotide analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2% deoxyribose or ribose, nucleobase polymers having positively charged sugar-guanidyl interlinkages such as those described in U.S. Pat. No. 6,013,785 and U.S. Pat. No. 5,696,253 (see also, Dagani 1995, Chem. & Eng. News 4-5:1153; Dempey et al., 1995, J. Am. Chem. Soc. 117:6140-6141). Such positively charged analogues in which the sugar is 2′-deoxyribose are referred to as “DNGs,” whereas those in which the sugar is ribose are referred to as “RNGs.” Specifically included within the definition of poly- and oligonucleotide analogs are locked nucleic acids (LNAs; see, e.g. Elayadi et al., 2002, Biochemistry 41:9973-9981; Koshkin et al., 1998, J. Am. Chem. Soc. 120:13252-3; Koshkin et al., 1998, Tetrahedron Letters, 39:4381-4384; Jumar et al., 1998, Bioorganic & Medicinal Chemistry Letters 8:2219-2222; Singh and Wengel, 1998, Chem. Commun., 12:1247-1248; WO 00/56746; WO 02/28875; and, WO 01/48190; all of which are incorporated herein by reference in their entireties).

“Polynucleotide or oligonucleotide mimic” refers to nucleobase polymers or oligomers in which the nucleobases are connected by a linkage other than a sugar-phosphate linkage or a sugar-phosphate analog linkage. Mimics with a specific linkage include peptide nucleic acids (PNAs) as described in any one or more of U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,451,968, 6,441,130, 6,414,112 and 6,403,763; all of which are incorporated herein by reference. Other types of mimics are described in the following publications: Lagriffoul et al., 1994, Bioorganic & Medicinal Chemistry Letters, 4: 1081-1082; Petersen et al., 1996, Bioorganic & Medicinal Chemistry Letters, 6: 793-796; Diderichsen et al, 1996, Tett. Lett. 37: 475-478; Fujii et al., 1997, Bioorg. Med. Chem. Lett. 7: 637-627; Jordan et al., 1997, Bioorg. Med. Chem. Lett. 7: 687-690; Krotz et al., 1995, Tett. Lett. 36: 6941-6944; Lagriffoul et al, 1994, Bioorg. Med. Chem. Lett. 4: 1081-1082; Diederichsen, U., 1997, Bioorganic & Medicinal Chemistry 25 Letters, 7: 1743-1746; Lowe et al., 1997, J. Chem. Soc. Perkin Trans. 1, 1: 539-546; Lowe et al., 1997, J. Chem. Soc. Perkin Trans. 11: 547-554; Lowe et al., 1997, I. Chem. Soc. Perkin Trans. 1 1:5 55-560; Howarth et al., 1997, I. Org. Chem. 62: 5441-5450; Altmann, K-H et al., 1997, Bioorganic & Medicinal Chemistry Letters, 7: 1119-1122; Diederichsen, U., 1998, Bioorganic & Med. Chem. Lett., 8:165-168; Diederichsen et al., 1998, Angew. Chem. mt. Ed., 37: 302-305; Cantin et al., 1997, Tett. Lett., 38: 4211-4214; Ciapetti et al., 1997, Tetrahedron, 53: 1167-1176; Lagriffoule et al., 1997, Chem. Eur. 1. '3: 912-919; Kumar et al., 2001, Organic Letters 3(9): 1269-1272; and the Peptide-Based Nucleic Acid Mimics (PENAMs) of Shah et al. as disclosed in WO 96/04000. All of which are incorporated herein by reference.

In some embodiments, a “peptide nucleic acid” or “PNA” can be an oligomer or polymer segment comprising two or more covalently linked subunits of the formula:

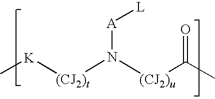

wherein, each J is the same or different and is selected from the group consisting of H, R¹, OR¹, NHR¹, NR¹₂, F, Cl, Br and I. Each K can be the same or different and be selected from the group consisting of O, S, NH and NR¹. Each R′ can be the same or different and can be an alkyl group having one to five carbon atoms that may optionally contain a heteroatom or a substituted or unsubstituted aryl group. Each A can be selected from the group consisting of a single bond, a group of the formula; —(CJ₂)ₛ- and a group of the formula; —(CJ₂)ₛC(O)—, wherein, J is defined above and each s can be a whole number from one to five. Each t can be 1 or 2 and each u can be 1 or 2. Each L can be the same or different and can be independently: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) or N8-(7-deaza-8-aza-adenine), other naturally occurring nucleobase analogs or other non-naturally occurring nucleobases.

In some other embodiments, PNAs are those in which the nucleobases are attached to an N-(2-aminoethyl)-glycine backbone, i.e., a peptide-like, amide-linked unit (see, e.g., U.S. Pat. No. 5,719,262; Buchardt et al., 1992, WO 92/20702; Nielsen et al., 1991, Science 254:1497-1500). A partial structure of a PNA that can be used in the methods and kits described herein, N-(2-aminoethyl)-glycine PNA, is illustrated in structure (I), below:

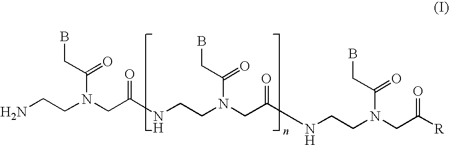

wherein:

n is an integer that defines the length of the N-(2-aminoethyl)-glycine PNA;

each B is independently a nucleobase; and

R is —OR′ or —NR′R′, where each R′ is independently hydrogen or (C₁-C₆) alkyl, preferably hydrogen.

“Chimeric Nucleobase Polymers or Chimeras” refers to a nucleobase polymer or oligomer comprising segments of different compositions. The first segment can comprise a single monomer or multiple monomers. For example, the first segment can comprise a single oligonucleotide or two or more oligonucleotides. Similarly, the second segment can comprise a single oligonucleotide analog, or two or more oligonucleotide analogs. Alternatively, the first segment can comprise one or more oligonucleotide analogs, and the second segment can comprise one or more oligonucleotide mimics.

“Label” refers to a moiety that, when attached to a probe, renders such a probe detectable using known detection methods, e.g., spectroscopic, photochemical, fluorescent, or electrochemiluminescent methods.

“Bind”, “Binding”, “Bound”, “Hybridize” or “Hybridization” are used interchangeably and refer to sequence specific base-pairing interactions of one nucleobase polymer with another nucleobase polymer that result in the formation of a double-stranded structure, a triplex structure, a quaternary structure or other higher order structure. Such base pairing interactions can occur via Watson-Crick base-pairing interactions, but can also be mediated by other hydrogen-bonding interactions, such as Hoogsteen base pairing.

Non-limiting examples of standard sequence-specific base pairing includes adenine base pairing with thymine or uracil and guanine base pairing with cytosine. Other non-limiting examples of other sequence specific base-pairing motifs include, but are not limited to, adenine base pairing with any of: 5-propynyl-uracil, 2-thio-5-propynyl-uracil, 2-thiouracil or 2-thiothymine; guanine base pairing with any of: 5-methylcytosine or pseudoisocytosine; cytosine base pairing with any of: hypoxanthine, N9-(7-deaza-guanine) or N9-(7-deaza-8-aza-guanine); thymine or uracil base pairing with any of: N9-(2-aminopurine), N9-(2-amino-6-chloropurine) or N9-(2,6-diaminopurine); and N8-(7-deaza-8-aza-adenine), being a universal base, base pairing with any other nucleobase, such as for example any of: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine) or N9-(7-deaza-8-aza-guanine) (See: Seela et al., Nucl. Acids, Res.: 28(17): 3224-3232 (2000)).

The above described sequence specific base pairing interactions include those pseudomonomer base pairing motifs as described by Haaima et al., 1997, Nucl. Acids Res., 25: 4639-4643; and Lohse et al. 1999, Proc. Natl. Acad. Sci. USA, 96: 11804-11808. Pseudomonomer base pairing is unique because N9-(2,6-diaminopurine) can be substituted for all adenine nucleobases and either 2-thiouracil and 2-thiothymine can be substituted for all thymine or uracil nucleobases in a probe. When probes are so configured, the probes of the probe sets containing these nucleobases cannot hybridize to form probe/probe complexes because they are pseudocomplementary, but can still hybridize to nucleobase sequences (See: Lohse at al. for a discussion of pseudocomplementary and pseudocomplementary nucleobases).

“Solid support” or “solid carrier” refers to any solid phase material upon which an oligomer is synthesized, attached, ligated or otherwise immobilized. Solid support encompasses terms such as “resin”, “solid phase”, “surface” and “support”. A solid support may be composed of organic polymers such as polystyrene, polyethylene, polypropylene, polyfluoroethylene, polyethyleneoxy, and polyacrylamide, as well as co-polymers and grafts thereof. A solid support may also be inorganic, such as glass, silica, controlled-pore-glass (CPG), or reverse-phase silica. The configuration of a solid support may be in the form of beads, spheres, particles, granules, a gel, or a surface. Surfaces may be planar, substantially planar, or non-planar. Solid supports may be porous or non-porous, and may have swelling or non-swelling characteristics. A solid support may be configured in the form of a well, depression or other container, vessel, feature or location. A plurality of solid supports may be configured in an array at various locations, addressable for robotic delivery of reagents, or by detection means including scanning by laser illumination and confocal or deflective light gathering.

“Support bound” means immobilized on or to a solid support. It is understood that immobilization can occur by any means, including for example; by covalent attachment, by electrostatic immobilization, by attachment through a ligand/ligand interaction, by contact or by depositing on the surface.

“Array” or “microarray” refers a predetermined spatial arrangement of oligomers present on a solid support or in an arrangement of vessels. Certain array formats are referred to as a “chip” or “biochip” (M. Schena, Ed. Microarray Biochip Technology, BioTechnique Books, Eaton Publishing, Natick, Mass. (2000). An array can comprise a low-density number of locations, e.g. 2 to about 12, medium-density, e.g. about a hundred or more locations, or a high-density number, e.g. a thousand or more. Typically, the array format is a geometrically regular shape that allows for fabrication, handling, placement, stacking, reagent introduction, detection, and/or storage. The array may be configured in a row and column format, with regular spacing between each location. Alternatively, the locations may be bundled, mixed or homogeneously blended for equalized treatment or sampling. An array may comprise a plurality of addressable locations configured so that each location is spatially addressable for high-throughput handling, robotic delivery, masking, or sampling of reagents, or by detection means including scanning by laser illumination and confocal or deflective light gathering.

### DESCRIPTION OF VARIOUS EMBODIMENTS

The methods and kits described herein find use in the multiplex analysis of nucleic acids. The methods and kits employ sets of sequence-specific probes that permit the repeated analysis of one or a plurality of nucleic acids in a single assay without having to use multiple samples. At least two probe sets are used, a first probe set, comprising probes having sequences that are at least partially complementary to sequences of interest, and a second probe set, comprising probes having sequences that are at least partially complementary and/or partially identical to the sequences of the first probe set.

The repeated analysis of one or a plurality of nucleic acids is achieved by using the second probe set to “strip” the first probe set from the nucleic acid sample being analyzed. In other words, the second probe set acts a “stripping agent” to remove or displace the first probe set from the sample being analyzed. Removal of the first probe set, allows for one or more additional probe sets to used to analyze the sample.

For example, an immobilized nucleic acid can be contacted with a first set of probes under conditions effective to permit sequence-specific binding between probes of the first set and sequences that are at least partially complementary thereto. The first probe set can comprise one probe, or two or more probes. In embodiments comprising two or more probes, the probe set can contain subsets. The probe subsets may comprise a single probe or a plurality of probes, each probe having a different sequence. The sequences of the subsets may be designed to be specific for the same nucleic acid, different nucleic acids, the same chromosome or for different chromosomes depending on the context in which the assay is to be used.

Following contact, unbound probes of the first set can be optionally removed and the nucleic acids can be monitored to determine whether any probes of the first set bound the nucleic acid. To determine whether any probes of the first set bound the nucleic acid, different, distinguishable, detectable labels can be used to label the probes. In other embodiments, the probes are unlabelled, and antibodies to nucleic acid duplexes can be used to determine if any probes of the first set bound the nucleic acid.

Once a determination has been made of whether any probes of the first set bound the nucleic acid, the nucleic acid can be contacted with a second set of probes under conditions effective to permit sequence-specific binding between probes of the second set and sequences that are at least partially complementary thereto. Conditions effective for permitting sequence-specific binding between probes of the second set and sequences that are at least partially complementary thereto can be determined experimentally by optimizing one or more of the factors commonly used to control stringency of hybridization until the desired degree of discrimination is achieved.

The number of probes in the second set can be less than, equal to, or greater than the number of probes in the first set. For example, in some embodiments, the number of probes in the second set is at least the sum of the number of probes in the first set. In some other embodiments, the second set might contain one probe that is identical in sequence to every probe in the first set, as well as one probe that is complementary to each probe in the first set such that the second set comprises at least two times the number of probes in the first set. In these embodiments, the probes of the second set that are both complementary and identical to a particular probe of the first set, are pseudo complementary with respect to each other, such that hybridization between probes of the second set does not occur.

The immobilized nucleic acid can then be contacted with a third set of probes under conditions effective to permit sequence-specific binding between probes of the third set and sequences that are at least partially complementary thereto. The third probe set can comprise one probe, or two or more probes. In embodiments comprising two or more probes, the third probe set can contain subsets. The probe subsets may comprise a single probe or a plurality of probes, each probe having a different sequence. The probes of the third set can be unlabeled or labeled with a different, distinguishable, detectable label. Following contact, unbound probes of the third set can be optionally removed and the nucleic acid can be monitored to determine whether any probes of the third set bound the nucleic acid.

Additional probes sets can be used to analyze the immobilized nucleic acid by contacting the nucleic acid with another probe set designed to strip the third probe set from the nucleic acid sample being analyzed. In this way, the nucleic acid can be re-analyzed numerous times for sequences of interest by repeatably contacting the nucleic acid with a probe set designed to hybridize to sequences of interest, and stripping that probe set with a probe set designed to function as a stripping agent.

Accordingly, provided herein are methods and kits suitable for the multiplex analysis of nucleic acids. In a multiplex assay, numerous conditions of interest are simultaneously or sequentially examined. Multiplex analysis relies on the ability to sort sample components or the data associated therewith, during or after the assay is completed. In performing a multiplex assay, different, distinguishable, detectable labels can be used to label two or more different probes that are to be used in an assay. By different, distinguishable, detectable labels we mean that it is possible to determine one label independently of, and sometimes in the presence of, the other label. The ability to differentiate between and/or quantitate each of the different, distinguishable, detectable labels facilitates the multiplexing of a hybridization assay because the data correlates with the hybridization of each distinct probe to a particular target sequence sought to be determined in the sample. Consequently, the multiplex assays can, for example, be used to simultaneously or sequentially detect, identify, quantitate and/or locate two or more target sequences in the same sample and in the same assay.

Thus, the methods and kits employ sets of sequence-specific probes to identify target sequences of interest. In some embodiments, the probe sets comprise at least a first set of probes and a second set of probes. In some other embodiments, the probe sets comprise a third set of probes. In still other embodiments, the probe sets comprise a fourth, fifth, sixth, seventh, eight, ninth, tenth, eleventh and/or twelfth set of probes. In still more embodiments, the number of sets of probes is greater than twelve.

A set of probes can comprise a single probe or more than one probe. For example, a set can comprise from 2 to 20 probes. In embodiments comprising two or more probes, the probe set can contain one or more subsets of probes. For example, there can be a single subset of probes, or there can be from 2 to 12 subsets of probes. In some embodiments, there can be from 1 to 7 subsets of probes, and in some other embodiments there can be from 1 to 4 subsets of probes. A subset can comprise a single probe or more than one probe. For example a subset of probes can comprise a single probe or from 2 to 20 probes.

The number of probes in a set or subset can be selected based upon a number of factors, such as the number of unique target sequences present in a nucleic acid sample or on the number of different detectable labels available for a given assay format. For example, Taneja et al., designed 30 probes for the X chromosome using publicly available data, but only 6 of the 30 probes were specific for sequences on the X chromosome. The remainder of the probes designed hybridized to the centromeric regions of multiple chromosomes. See, Tanega et al., 2001, Genes, Chromosomes & Cancer, 30:57-63. Thus, only 6 of the 30 probes could be used to detect the X chromosome.

Generally, two types of probe sets are used in the methods and kits described herein. The first type, referred to herein as the “first set”, “third set”, “fifth set”, seventh set”, etc., “signal probe set”, or “signal probes” can be designed to detect target sequences of interest. Target sequences of interest are detected by monitoring the nucleic acid sample to determine whether any of the signal probes bound their respective target sequence of interest, i.e. detecting the presence of a hybridization complex comprising a signal probe and a target sequence of interest. As used herein, detecting can be used to quantitate, identify, determine the presence of hybridization complexes in a nucleic acid sample of interest, or the location of hybridization complexes of a nucleic acid of interest. As described below, the complexes can be detected using antibodies that recognize nucleic acid duplexes or by labeling the signal probes with a distinguishable, detectable label.

The nucleobase sequence of each of the signal probes can be designed to be complementary, substantially complementary, or partially complementary to a different target sequence as it has been demonstrated that greater sequence discrimination can be obtained when utilizing probes that comprise one or more point mutations (base mismatch) between the probe and the target sequence (See: Guo et al., Nature Biotechnology 15:331-335 (1997)). By “substantially complementary” we mean that the probe comprises a sequence that produces a single mismatch within the probe/target sequence complex. By “partially complementary” we mean that the probe comprises a sequence that produces two or more mismatches within the probe/target sequence complex.

The second type of probe set used in the methods and kits described herein can be used to “strip” the signal probes sets from the nucleic acid sample. The second type of probes sets are referred to herein as the “second set”, “fourth set”, “sixth set”, etc., “competitor set”, “competitor probes”, or “stripping agents”. The competitor probe sets can be labeled or unlabeled.

In some embodiments, the competitor probe set can all be at least partially identical to the first set of probes. In some other embodiments, the competitor probe set can be all at least partially complementary to the first set of probes. In still some other embodiments, some of the probes of the competitor probe set can be at least partially identical to some of the probes of the first set and the remainder of the probes of the competitor probe set can be at least partially complementary to the sequences of the remainder of the probes of the first set. In still some other embodiments, there can be in the competitor probe set a probe that is at least partially identical to each probe of the first set and a probe that is at least partially complementary to each probe of the first set. It should be apparent therefore, that there are many possible embodiments for the competitor probe set and that the number of probes of the competitor probe set is not absolutely set by the number of probes of the first set of probes. Generally the number of probes in the competitor probe set is greater than or equal to the sum of the probes in the first probe set. For example, if there are 20 probes in the first set, the second set can contain 20 or more probes.

When referring to competitor probes by “identical” we mean that the sequence of the competitor probe is the same as the sequence of the corresponding signal probe (e.g. the first probe set) of the previously used signal probe set. When referring to competitor probes by “substantially identical” we mean that the competitor probe comprises one nucleobase that differs from the sequence of a signal probe (e.g. the first probe set) of the previously used signal probe set. When referring to competitor probes, by “partially identical” we mean that the competitor probe comprises two or more nucleobases that differ from the sequence of a signal probe (e.g. the first probe set) of the previously used signal probe set.

When referring to competitor probes by “complementary” we mean that the competitor probe forms a hybrid with a signal probe of the previously used set of signal probes (e.g. the first probe set), said hybrid comprising no base-pairing mismatches. When referring to competitor probes by “substantially complementary” we mean that the competitor probe forms a hybrid with a signal probe of the previously used set of signal probes (e.g. the first probe set), said hybrid comprising a single base-pairing mismatch. When referring to competitor probes by “partially complementary” we mean that the competitor probe forms a hybrid with a signal probe (e.g. the first probe set), of the previously used set of signal probes, said hybrid comprising two or more base-pairing mismatches.

Mixing competitor probes within a set wherein one probe is at least partially identical to a target sequence and one probe is at least partially complementary to a target sequence is not a problem when the probes comprise pseudocomplementary nucleobases (see, e.g., Haaima et al., 1997, Nucleic Acids Research, 25(22):4639-4643, and Lohse et al., 1999, PNAS, 96(21):11804-11808, both of which are incorporated herein by reference in their entirety). Probes comprising pseudocomplementary nucleobases do not hybridize to each other. Instead, pseudocomplementary probes hybridize to a complementary, substantially complementary, or partially complementary target sequence, or to a complementary, substantially complementary, or partially complementary signal probe.

The second set of probes can be used to “strip” the signals generated from the first set of probes. By “strip”, “stripped” or “stripping” we mean that the signal associated with a set of signal probes is either diminished or eliminated, such that the signal is no longer detectable. As used in the context of the present teachings, the process of “stripping” requires the use of competitor probes, and hence, is not merely associated with an increase in temperature and/or the use of washing steps.

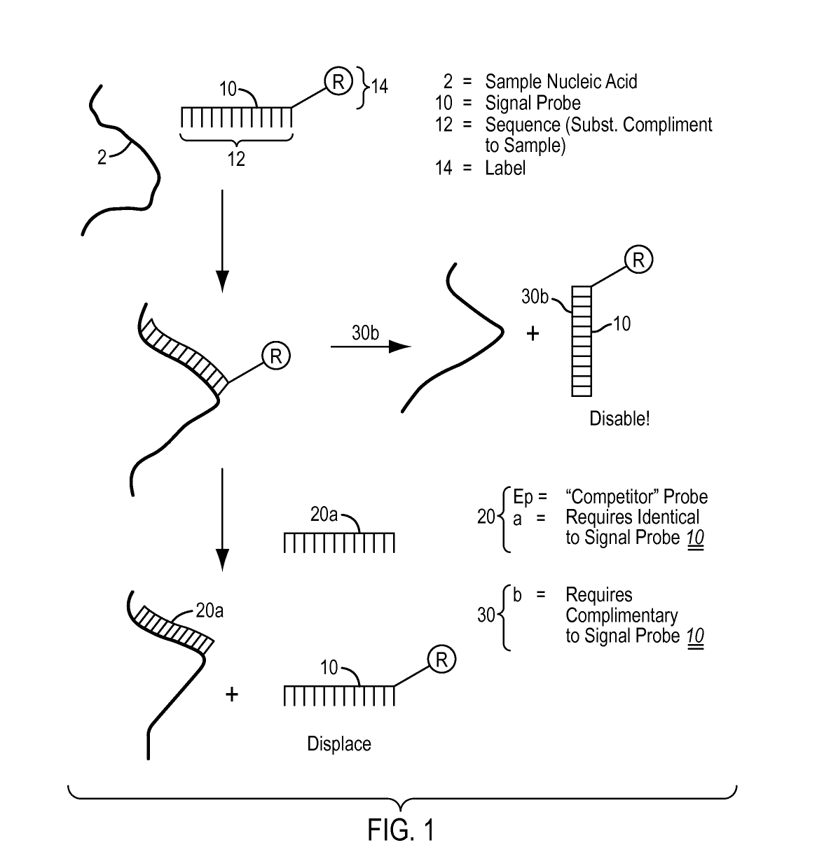

Without intending to be bound to any theory or mechanism, FIG. 1 illustrates two possible mechanisms by which competitor probes can strip the signals associated with labeled signal probes. As illustrated in FIG. 1, one possible mechanism is displacement of signal probe 10 from nucleic acid 2 by competitor probe 20 that has a sequence that is at least partially identical to signal probe 10. As shown in FIG. 1, competitor probe 20 binds to the same target sequence on nucleic acid 2 as does signal probe 10. It can be said that competitor probe 20 displaces signal probe 10 from nucleic acid 2 by binding nucleic acid 2 when an equilibrium exists between the binding of signal probe 10, or competitor probe 20 to the target sequence. As separately illustrated in FIG. 1, a second possible mechanism can also explain the stripping of the signal associated with signal probe 10. In this case, competitor probe 30 has a sequence that is at least partially complementary to signal probe 10. As illustrated in FIG. 1, competitor probe 30 binds to signal probe 10 when equilibrium has, by breathing of the hybrid, caused signal probe 10 to at least partially dissociate from the target sequence. Formation of the hybrid between the signal probe and the competitor probe can be said to disable the signal probe because it inhibits the signal probe 10 from again binding nucleic acid 2. To prevent or minimize re-hybridization of signal probes with the target sequence, the concentration of competitor probes can be adjusted to be greater than the concentration of the signal probes still present in the assay; for example where the excess signal probes have been washed away. Regardless of the mechanism, the net effect of using competitor probes is to strip the signals associated with signal probes.

Competitor probe sets can be added after the probe/target sequence complex has been formed and the signals detected, or competitor probe sets and signal probe sets can be added together. For example, the second set of competitor probes can be added after the first set of signal probes. However, the third set of signal probes can be added at the same time as the second set of competitor probes, or the third set of signal probes can be added after the nucleic acid sample has been contacted with the second set of competitor probes.

Once the signals from the first probe set have been stripped, the sample can be re-examined with one or more additional probe sets to determine one or more additional sequences of interest. Because the signals from the first probe set have been stripped, the same distinguishable, detectable labels applied to the first probe set can be reused in a subsequent signal probe set to again extract information from the sample.

For example, a third set of probes can be added to extract additional information from the sample. The third set of probes can be much like the first set of probes. The third set of probes can be referred to as signal probes, a set of signal probes, or a second set of signal probes. The nucleobase sequence of each of the probes of the third set of probes can be designed to be complementary, substantially complementary, or at least partially complementary to a different target sequence useful in determining a sequence of interest in a sample. The formation of a hybridization complex between a probe of the third set and a sequence of interest can be detected by incorporating distinguishable detectable labels into the probes of the third set, or by the addition of antibodies that recognize nucleic acid duplexes.

In-so-far as the third set of probes functions like the first set of probes, albeit usually designed to determine sequences of interest that differ from those determined with the first set of probes, signals from the third set of probes can be stripped using a fourth set of competitor probes that perform much like the second set of competitor probes provided that the probes of the fourth set are designed to be at least partially identical and/or at least partially complementary to probes of the third set. In this way, the process of determining unique target sequences of interest with the first set of probes is repeated with the third set of probes, and stripping the signals associated with the first set of probes using a second set of probes is repeated for the third set of probes by using the forth set of probes. Accordingly, the process of determining target sequence information with one set of probes and stripping the target sequence information with a subsequent set of probes can be repeated (cycled) numerous times to generate large amounts of data from a single sample. Furthermore, this method can be performed in multiplex mode wherein probes of a set comprise different, distinguishable, detectable labels in a manner that greatly expands the amount of information attainable from a single sample.

Because the final set of signals need not be stripped, a cycle of repetition of this process can be defined with respect to first repeating the step of stripping the signal associated with a set of probes followed by a step of detecting the signal associated with another set of probes. This cycle of stripping and detecting can be performed numerous times. It can be repeated 1 time, 2 times, 3 times, or 4 or more times, following the addition of the first probe set. In some embodiments, it can be performed from 1 to 4 additional times. In some other embodiments, it can be performed from 1 to 8 additional times.

Thus, the methods and kits described herein can be used to detect a target sequence of interest. As used herein “target sequence” or “sequences of interest” refer to sequences associated with a particular nucleobase, genotype, phenotype, disease state, or organism. Target sequences can also be used to detect an abnormality, such as an abnormality in chromosome number. A target sequence to be determined is associated with a nucleobase polymer. For example, the target sequence can exist as part of a nucleobase oligomer, polynucleotide or oligonucleotide, polynucleotide or oligonucleotide analog, polynucleotide or oligonucleotide mimic, or chimeric oligomer (i.e. a nucleobase polymer). The target sequence can be the whole of the nucleobase polymer or it can be a subsequence of the nucleobase polymer. The target sequence can be optimally chosen to be directed to a unique nucleobase sequence within the sample. It is to be understood that the nature of the target sequence is not a limitation of the various embodiments described herein.

The nucleobase polymer can be a nucleic acid. Nucleic acid to be analyzed can be from any source. For example, nucleic acid can be isolated or enriched from a sample (e.g. by amplification). In some other embodiments, the nucleic acid to be analyzed can be present in an immobilized cell, tissue or in a chromosome in metaphase or interphase state. By “chromosome” herein is meant the self-replicating genetic material of a prokaryotic or eukaryotic cell. In some other embodiments, the nucleic acid, whether or not first amplified, can be isolated and immobilized.

If the nucleic acid is isolated from a sample, the sample containing the nucleic acid can be provided from nature or it can be synthesized or supplied from a manufacturing process. For example, nucleic acid can be produced from an amplification process, contained in a cell or organism, or otherwise be extracted from a cell or organism. Examples of amplification processes that can be the source for the target sequence include, but are not limited to, Polymerase Chain Reaction (PCR), Ligase Chain Reaction (LCR), Strand Displacement Amplification (SDA; see, e.g., Walker et al., 1989, PNAS 89:392-396; Walker et al., 1992, Nucl. Acids Res. 20(7):1691-1696; Nadeau et al., 1999, Anal. Biochem. 276(2):177-187; and U.S. Pat. Nos. 5,270,184, 5,422,252, 5,455,166 and 5,470,723), Transcription-Mediated Amplification (TMA), Q-beta replicase amplification (Q-beta), Rolling Circle Amplification (RCA), Lizardi, 1998, Nat. Genetics 19(3):225-232 and U.S. Pat. No. 5,854,033), or Asynchronous PCR (see, e.g., WO 01/94638).

Nucleic acids, whether or not amplified, can be analyzed by immobilizing them to the surface of a solid support using the well-known process of UV-crosslinking. Alternatively, the nucleic acids can be covalently bound to a surface of a solid support by the reaction of a suitable functional group on the nucleic acid. Methods are well known in the art for the attachment of nucleic acids to surfaces, see, e.g., U.S. Pat. No. 6,100,676, the disclosure of which is incorporated herein by reference. In some embodiments, the nucleic acids can be immobilized to a surface to form an array.

Nucleic acid samples that do not exist in a single-stranded state in the region of the target sequence(s) can be rendered single-stranded in such region(s) prior to detection or hybridization. For example, nucleic acid samples can be rendered single-stranded in the region of the target sequence using heat denaturation. For polynucleotides obtained via amplification, methods suitable for generating single-stranded amplification products can be used. Non-limiting examples of amplification processes suitable for generating single-stranded amplification product polynucleotides include, but are not limited to, T7 RNA polymerase nm-off transcription, RCA, Asymmetric PCR (Bachmann et al., 1990, Nucleic Acid Res., 18, 1309), and Asynchronous PCR (WO 01/94638). Commonly known methods for rendering regions of double-stranded polynucleotides single stranded, such as the use of PNA openers (U.S. Pat. No. 6,265,166), may also be used to generate single-stranded target sequences within a double stranded polynucleotide.

Nucleic acids can also be analyzed in fixed cells, tissues, chromosomes and microorganisms. To analyze the nucleic acid (including chromosomal nucleic acid) present in a fixed cell, tissue, chromosome or microorganism, the sample can be obtained from various sources such as from, clinical samples, environmental samples, industrial samples, research samples, individual donors or from pre-implantation embryos associate with in-vitro fertilization techniques. Cells that can be obtained from individual donors include, without limitation, lymphoblasts, fibroblasts, peripheral blood mononuclear cells, and hemopoietic progenitor cells. When cells are obtained from pre-implantation embryos, the embryos can be dissociated to obtain individual cells for analysis (see, e.g., Clouston et al., 1997, Hum. Genet. 101:30-36; Munné et al., 1998, Molecular Human Reproduction, 4(9):863-870) or the cells, i.e. blastomeres, removed by micro-manipulation (see, e.g., Munné et al., 1993, Human Reproduction, 8(12):2185-2191). Regardless of the source, the cells, tissues, chromosomes or microorganisms can be fixed on a solid support, such as a glass slide, and analyzed. See for example: Taneja et al., 2001, Genes, Chromosomes & Cancer, 30:57-63; Clouston et al., 1997, Hum. Genet., 101:30-36; Perry-O'Keefe, et al., 2001, Journal of Microbiological Methods, 47:281-292; Sender, et al., 2002, Journal of Microbiological Methods, 38:1-17; and, Williams et al., PNA Fluorescent In Situ Hybridization for Rapid Microbiological and Cytogenetic Analysis, Chapter 12, In Methods in Molecular Biology, vol. 208, Peptide Nucleic Acids Methods and Protocols, edited by Peter E. Nielsen, Humana Press, Totowa, N.J.

As discussed above, two types of probe sets can be used in the method and kit embodiments described herein. One type of probe set is the signal probe set and the other probe set type is the competitor probe set. Examples of signal probe sets thus far described are the first probe set and the third probe set. The signal probes can be unlabeled or labeled. In most embodiments, the signal probes comprise a label and are sometimes referred to herein as “labeled probes” or “labeled probe sets”.

The competitor probes will typically be unlabeled and are sometimes referred to herein as “unlabeled probes”, or “unlabeled probe sets”. Examples of competitor probe sets thus far described are the second probe set and the fourth probe set. In some embodiments, it may be desirable for the competitor probe set to comprise a label. For example, when used for quality control purposes, the competitor probes can be optionally labeled with detectable labels that are distinguishable from the labeled signal probes of the immediately preceding signal probe set.

Labeled probes comprise a reporter or a signal label capable of producing a detectable signal when the labeled probe is hybridized to a target sequence. A labeled probe can comprise a label that is attached directly to the probe and is detectable or produces a detectable signal. The labels may be attached to the labeled probes at virtually any position. Thus, the labels may be attached to a terminus, to a terminal or internal nucleobase or to the backbone. Although the type of label is not critical to success, the labels used should produce detectable signals. When multiplex assays are utilized the various detectable labels of a set of probes should be different and distinguishable. By “distinguishable” we mean that the labels should be spectrally resolvable from one another.

As discussed below, the number of labels used in the signal probe sets can depend on the number of spectrally resolvable labels available and the labeling method (see, e.g., Munné and Cohen, 1998, Human Reproduction Update, 4(6):842-855). For example, if fluorophores are used to label the signal probes, between 1 to 7 fluorophores are available as labels for the signal probes. In contrast, if quantum dots are used to label the signal probes, the number of spectrally resolvable labels can vary from 1 to 24, or more than 24 depending on the assay conditions.

The labeled probe can comprise a label that is a fluorophore. Non-limiting examples of fluorophores suitable for labeling probes used in the method and kit embodiments described herein include Spectrum-Orange™, Spectrum-Green™, Spectrum-Aqua™, Spectrum-Red™, Spectrum-Blue™, Spectrum-Gold™, fluorescein isothiocyanate, rhodamine, and FluoroRed™, 5(6)-carboxyfluorescein (Flu), 6-((7-amino-4-methylcoumarin-3-acetyl)amino)hexanoic acid (Cou), 5(and 6)-carboxy-X-rhodamine (Rox), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye (Cyanine dyes 2, 3, 3.5, 5 and 5.5 are available as NHS esters from Amersham, Arlington Heights, Ill.) or the Alexa dye series (Molecular Probes, Eugene, Oreg.).

In embodiments using spectrally resolvable fluorophores, the number of subsets used per labeled probe set can vary from 1 to 7. Preferably, from 1 to 4 subsets are used per set of labeled signal probes.

Other non-limiting examples of detectable moieties (labels) suitable for labeling signal probes include dextran conjugates, branched nucleic acid detection systems, spin labels, radioisotopes, haptens, and acridinium esters. Non-limiting examples of enzymes include polymerases (e.g. Taq polymerase, Klenow PNA polymerase, T7 DNA polymerase, Sequenase, DNA polymerase 1 and phi29 polymerase), alkaline phosphatase (AP), horseradish peroxidase (HRP), soy bean peroxidase (SBP)), ribonuclease and protease. Non-limiting examples of haptens include 5(6)-carboxyfluorescein, 2,4-dinitrophenyl, digoxigenin, and biotin. Other suitable labeling reagents and methods of attachment would be recognized by those of ordinary skill in the art of PNA, peptide or nucleic acid synthesis.

In some embodiments, it may be desirable for a labeled probe to be a self-indicating probe. For example, “molecular beacon” or “hairpin self-indicating” probes can be used in the methods and kits described herein. By “hairpin” is meant a construct comprising a single-stranded loop region and a double-stranded stem region. By self-indicating we mean that the signal from at least one label of the probe changes depending upon whether or not the probe exists in a hybridized state as compared with an unhybridized state. A hairpin self-indicating probe can be designed to have a donor molecule on one end and an acceptor molecule on the other. When unhybridized the acceptor molecule quenches the detectable signal produced by the donor molecule. However, when the hairpin self-indicating probe hybridizes to a target sequence, a distance too great separates the donor and acceptor for efficient energy transfer. Accordingly, the acceptor no longer efficiently quenches the signal produced by the donor. Thus, the hairpin probe can be said to be “off” when unhybridized and can be said to be “on” when hybridized to a target sequence.

Hairpin self-indicating probes are known in the art (see, e.g., Tyagi et al., 1996, Nature Biotechnology 14:303-308; Nazarenko et al., 1997, Nucl. Acids Res. 25:2516-2521 for reviews see: Tan et al., 2000, Chem. Eur. J. 6:1107; Fang et al., 2000, Anal. Chem. 72:747 A; all of which are incorporated herein by reference) and have a nucleobase sequence capable of adopting a hairpin conformation in solution. In a specific embodiment, the hairpin probes can include a FRET donor on one end (e.g., 3′-terminus) and a FRET acceptor at the other end (e.g., 5′-terminus) such that when the probe is in the hairpin conformation, the FRET acceptor quenches the detectable signal produced by the FRET donor. In other embodiments, non-FRET donor and acceptors can be used (see U.S. Pat. No. 6,150,097; incorporated herein by reference). A hairpin self-indicating probe can be made entirely from PNA (see U.S. Pat. No. 6,355,421; incorporated herein by reference in its entirety).

Linear self-indicating probes may also be used in the method and kit embodiments described herein. As used herein, “linear” refers to a probe that assumes a conformation that is not a hairpin conformation. However, the term “linear” is not intended to imply that the probe does not contain secondary or tertiary structure. Thus, a linear self-indicating probe may be linear or may assume a conformation that is not a hairpin conformation. Like hairpin probes, linear self-indicating probes include a donor and an acceptor. Also like hairpin probes, linear self-indicating probes can remain substantially quenched until hybridized to a target sequence. A variety of different types of linear self-indicating probes suitable for use as self-indicating probes are known in the art, and include, by way of example and not limitation, the dual-label DNA probes commonly referred to in the art as TaqMan® probes (see U.S. Pat. Nos. 5,210,015, 6,258,569, and 6,503,720); the dual-label PNA probes described in Kuhn et al., 2002, J. Am. Chem. Soc. 124(6):1097-1103 (as well as the references cited therein), and are also described in U.S. Pat. No. 6,485,901 (as well as the references cited therein), the disclosures of which are incorporated herein by reference. Linear self-indicating probes can comprise either FRET or non-FRET pairs of donor and acceptor moieties.

Another type of self-indicating probe comprises only one label. Although it does not comprise a donor and acceptor moiety, the signal from that label changes based upon whether or not the probe is hybridized to a target sequence. One example of such a self-indicating is described in U.S. Pat. No. 6,329,144, herein referred to as an “intercalating self-indicating probe”. These intercalating self-indicating probes incorporate a reporter group that interacts with the nucleic acid to thereby produce a detectable signal upon hybridization. Accordingly, the label has little or no detectable signal until the probe hybridizes to the target sequence.

Regardless of the type, self-indicating probes can be used in the method and kit embodiments where it might be advantageous to use probes that change properties depending upon their hybridization state. For example, in certain assays it may be desirable to use probe sets comprising self-indicating probes because it may be difficult or undesirable to wash away unhybridized probe prior to determining signal associated with the probe/target sequence complex formation. When using self-indicating probes this is not necessarily a problem because the unhybridized probes can be distinguished from the hybridized probes. Consequently, the use of self-indicating probes can render optional the removal of excess signal probe at certain steps of an assay. For example it may be optional to wash away excess signal probe before determining signal associated with probe/target sequence complex formation.

In some embodiments, the label can be a microsphere comprising a spectral code, commonly referred to in the art as a “quantum dot” (see U.S. Pat. No. 6,500,622, the disclosure of which is incorporated herein by reference). The spectral code can comprise one or more semiconductor nanocrystals, having at least one different fluorescent characteristic, for example excitation wavelength, emission wavelength, emission intensity, etc. By attaching the signal probes to quantum dots having a range of distinguishable spectra allows for the simultaneous analysis of more sequences than is currently possible using existing fluorophores. For example, 12 or more spectrally resolvable labels can be used in a single assay. Such label formats are particularly well suited for use in multiplex assays because of the tremendous diversity of different, distinguishable, detectable labels.

The number of spectrally resolvable labels used in a single assay can also be increased by using combinatorial or ratiometric labeling. In combinatorial labeling, the number of all possible combinations is described by the formula X=2ⁿ−1, where n refers to the number of labels used. Using three fluorescent-labeled nucleotides (FITC-dUTP, Cy3-dUTP and AMCA-dUTP), seven different DNA probes can be labeled and simultaneously identified after hybridization, based on color combinations. For example, a DNA probe labeled with FITC will fluoresce green, another one labeled with AMCA will fluoresce blue, whereas a third one labeled with FITC and AMCA will fluoresce cyan. Similarly, combing probes in which one probe is labeled red and the other with green yields a yellow signal, the combination of a blue and a red labeled probes yields a magenta signal, whereas the combination of probes in which one probe is labeled with FITC-green, another is labeled with AMCA-blue and a third is labeled with Cy3-orange/red fluoresces “white”.

If ratio labeling is used, in theory many targets can be distinguished with a few labels. With ratio labeling, a mixture of probes is used wherein each probe is labeled with a resolvable label. The amount of each probe used in the mixture is at a set ratio to one another. Each target is distinguished by possessing different ratios of the colors used. For example, using two labels, red and green, a first target can be detected using only red labeled probes (i.e. target appears red), a second target can be detected using only green labeled probes (i.e. target appears green), a third target can be detected using a mixture of a red labeled probes and green labeled probes at a ratio of 75:25, such that the third target is distinguished from the first target based on the shade of red observed (i.e., the third target will be a less intense shade of red), a fourth target can be detected using a mixture of a red labeled probes and green labeled probes at a ratio of 65:35, such that the fourth target is distinguished from the first and third targets, again based on the shade of red observed (i.e., the fourth target appears orange), a fifth target can be detected using a mixture of a red labeled probes and green labeled probes at a ratio of 50:50, such that the fifth target is appears yellow, and so forth. Computer software is often required to sufficiently distinguish the different ratios.

In some embodiments, the signal probe sets do not comprise a detectable label. In these embodiments, an intrinsic feature of the probe or the complex formed between the probe and the target sequence, rather than an added label, can be exploited for this purpose. For example, antibodies that specifically recognize RNA/DNA duplexes have been demonstrated to have the ability to recognize probes made from RNA that are bound to DNA targets (Rudkin and Stollar, 1977, Nature, 265:472-473). Another example of a means to visualize the bound probe when the nucleic acid sequences in the probe do not directly carry some modified constituent is the use of antibodies to thymidine dimers (Nakane et al., 1987, Acta Histochem. Cytochem., 20 (2): 229), illustrate a method using thymine-thymine dimerized DNA as a marker for in situ hybridization. See also U.S. Pat. No. 6,607,877. Similarly antibodies that specifically recognize LNA/DNA, or PNA/DNA can be used in the methods and kits described herein. See U.S. Pat. No. 5,612,458.

The probes of the probe sets may be synthesized using routine methods. For example, methods of synthesizing oligonucleotide probes are described in U.S. Pat. No. 4,973,679; Beaucage, 1992, Tetrahedron 48:2223-2311; U.S. Pat. No. 4,415,732; U.S. Pat. No. 4,458,066; U.S. Pat. No. 5,047,524 and U.S. Pat. No. 5,262,530; all of which are incorporated herein by reference. The synthesis may be accomplished using automated synthesizers available commercially, for example the Model 392, 394, 3948 and/or 3900 DNA/RNA synthesizers available from Applied Biosystems, Foster City, Calif. Similarly, methods of synthesizing labeled oligonucleotide probes are also well-known. As a specific example, see WO 01/94638 (especially the disclosure at pages 16-21), the disclosure of which is incorporated herein by reference.

Methods of synthesizing labeled and unlabeled oligonucleotide analog probes are also well-known. See for example U.S. Pat. No. 6,479,650 and U.S. Pat. No. 6,432,642, both of which are incorporated herein by reference in their entirety.

Methods for the chemical assembly of PNAs are well known (see U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,201,103, 6,350,853, 6,357,163, 6,395,474, 6,414,112, 6,441,130, 6,451,968; all of which are herein incorporated by reference; also see PerSeptive Biosystems Product Literature). As a general reference for PNA synthesis methodology see Nielsen et al., Peptide Nucleic Acids; Protocols and Applications, Horizon Scientific Press, Norfolk England (1999).

Chemicals and instrumentation for the support bound automated chemical assembly of peptide nucleic acids are now commercially available. Both labeled and unlabeled PNA oligomers are likewise available from commercial vendors of custom PNA oligomers. Chemical assembly of a PNA is analogous to solid phase peptide synthesis, wherein at each cycle of assembly the oligomer possesses a reactive alkyl amino terminus that is condensed with the next synthon to be added to the growing polymer.

PNA may be synthesized at any scale, from submicromole to millimole, or more. PNA can be conveniently synthesized at the 2 μmole scale, using Fmoc(Bhoc) protecting group monomers on an Expedite Synthesizer (Applied Biosystems) using a XAL, PAL or many other commercially available peptide synthesis supports. Alternatively, the Model 433A Synthesizer (Applied Biosystems) with a suitable solid support (e.g. MBHA support) can be used. Moreover, many other automated synthesizers and synthesis supports can be utilized. Synthesis can be performed using continuous flow method and/or a batch method. PNA can be manually synthesized.

Regardless of the synthetic method used, because standard peptide chemistry is utilized, natural and non-natural amino acids can be routinely incorporated into a PNA oligomer. For the purposes of the design of a hybridization probe suitable for antiparallel binding to the target sequence, the N-terminus of the probing nucleobase sequence of the PNA probe is the equivalent of the 5′-hydroxyl terminus of an equivalent DNA or RNA oligonucleotide.

Non-limiting methods for labeling PNAs are described in U.S. Pat. Nos. 6,110,676, 6,280,964, 6,355,421, 6,485,901, 6,361,942, and 6,441,152 (all of which are herein incorporated by reference), or are otherwise well known in the art of PNA synthesis and peptide synthesis. Methods for labeling PNA are also discussed in Nielsen et al., Peptide Nucleic Acids; Protocols and Applications, Horizon Scientific Press, Norfolk, England (1999).

The synthesis, labeling and modification of PNA chimeras can utilize methods known to those of skill in the art as well as those described above. A suitable reference for the synthesis, labeling and modification of PNA chimeras can be found in WIPO published patent application number WO 96/40709, now issued as U.S. Pat. No. 6,063,569, incorporated herein by reference in its entirety. Moreover, the methods described above for PNA synthesis and labeling often can be used for modifying the PNA portion of a PNA chimera. Additionally, well known methods for the synthesis and labeling of nucleic acids can often be used for modifying the oligonucleotide portion of a PNA chimera. Exemplary methods can be found in U.S. Pat. No. 5,476,925, U.S. Pat. No. 5,453,496, U.S. Pat. No. 5,446,137, U.S. Pat. No. 5,419,966, U.S. Pat. No. 5,391,723, U.S. Pat. No. 5,391,667, U.S. Pat. No. 5,380,833, U.S. Pat. No. 5,348,868, U.S. Pat. No. 5,281,701, U.S. Pat. No. 5,278,302, U.S. Pat. No. 5,262,530, U.S. Pat. No. 5,243,038, U.S. Pat. No. 5,218,103, U.S. Pat. No. 5,204,456, U.S. Pat. No. 5,204,455, U.S. Pat. No. 5,198, U.S. Pat. No. 540, U.S. Pat. No. 5,175,209, U.S. Pat. No. 5,164,491, U.S. Pat. No. 5,112,962, U.S. Pat. No. 5,071,974, U.S. Pat. No. 5,047,524, U.S. Pat. No. 4,980,460, U.S. Pat. No. 4,923,901, U.S. Pat. No. 4,786,724, U.S. Pat. No. 4,725,677, U.S. Pat. No. 4,659,774, U.S. Pat. No. 4,500,707, U.S. Pat. No. 4,458,066 and U.S. Pat. No. 4,415,732, all of which are incorporated herein by reference in their entireties.

The probes of the probe sets used in the method and kit embodiments described herein can be virtually any nucleobase oligomer that is capable of binding to a target sequence in a sequence-specific manner. Thus, probes include, but are not limited to oligonucleotides, oligonucleotide analogs, oligonucleotide mimics, and chimeras, as defined above. Nucleobase polymers with rapid hybridization kinetics can be used, such that under the conditions of the assay, hybridization between a probe and the target sequence occurs within approximately thirty minutes. Examples of suitable nucleobase polymers include oligonucleotide mimics (i.e. PNA), oligonucleotide analogs (i.e. LNA), and/or chimeras (i.e. PNA linked to LNA, LNA linked to DNA, or LNA linked to RNA). The nucleobase polymer can be a DNA or an RNA.

Although in many instances, all of the probes of the sets and subsets will be of the same chemical composition, they need not be. Accordingly, in some embodiments, all of the signal probes and competitor probes can be PNA probes. In some other embodiments, some of the signal probes and/or some of the competitor probes can be PNA probes, while other signal probes and competitor probes can be DNA, RNA, LNA or chimeras. In still other embodiments, some of the signal probe can be DNA and some RNA whilst some of the competitor probes are PNA and some of the competitor probes are LNA. It should therefore be apparent that the composition of the probes of the probes sets is not fixed to one type of probe and indeed the probe type (e.g. PNA, LNA, RNA, DNA and chimera) can be mixed and matched within probe sets as desired. Accordingly, this is not a limitation of the method and kit embodiments described herein.

Those of ordinary skill in the art of hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for forming a hybrid combination can be found by the well-known technique of fixing several of the aforementioned stringency factors and then determining the effect of varying a single stringency factor. The same stringency factors can be modulated to thereby control the stringency of hybridization of a PNA to a nucleic acid, except that the hybridization of a PNA is fairly independent of ionic strength. Optimal or suitable stringency for an assay can be experimentally determined by examination of each stringency factor until the desired degree of discrimination is achieved.

The probes of the probe sets of the method and kit embodiments can form hybrids under suitable hybridization conditions. Generally, the more closely related the background causing nucleic acid contaminates are to the target sequence, the more carefully stringency will be controlled. Blocking probes can also be used as a means to improve discrimination beyond the limits possible by mere optimization of stringency factors (See: Fiandaca et al. “PNA Blocker Probes Enhance Specificity In Probe Assays”, Peptide Nucleic Acids: Protocols and Applications, pp. 129-141, Horizon Scientific Press, Wymondham, UK, 1999; herein incorporated by reference). Typically, the blocking probes are PNA probes (See: Coull et al., U.S. Pat. No. 6,110,676, herein incorporated by reference). Suitable hybridization conditions will thus comprise conditions under which the desired degree of discrimination is achieved such that an assay generates an accurate (within the tolerance desired for the assay) and reproducible result. Often this is achieved by adjusting stringency until sequence specific hybridization is achieved. In some embodiments, it may be preferable to perform the assay under denaturing conditions. For example, the assay can be performed under low salt (e.g. less than 100 mM total ionic strength), in the presence of formamide, immediately after heat denaturation, or any combination of the foregoing. Nevertheless, aided by no more than routine experimentation and the disclosure provided herein, those of skill in the art will be able to determine suitable hybridization conditions for performing the assays described herein.

FIG. 2A-2E illustrate various embodiments of the methods described herein. FIG. 2A illustrates the use of two sets of labeled signal probes and one set of unlabeled competitor probes having sequences that are at least partially identical to the labeled signal probes to detect sequences of interest in an immobilized nucleic acid. In the embodiment illustrated in FIG. 2A, nucleic acid sample 2 is contacted with a first set of labeled signal probes comprising subsets s1, s2, s3, and s4 under conditions effective to permit sequence-specific binding between probes 10a, 10b, 10c, and 10d and sequences present on nucleic acid 2 that are at least partially complementary thereto. Each subset bears a different distinguishable fluorophore label, i.e., subset s1 has label R, subset s2 has label Y, subset s3 has label B, and subset s4 has label G. An optional wash step can be used to remove any unbound labeled signal probes, i.e. signal probe 10d, prior to determining whether any of the labeled signal probes, i.e. label probes 10a, 10b, and 10c, bound nucleic acid 2.

Following a determination of whether label probes 10a, 10b, or 10c bound nucleic acid 2, nucleic acid 2 is contacted with a second set of probes, i.e. unlabeled competitor probes 20a, 20b, 20c, and 20d, under conditions effective to permit sequence-specific binding between probes of the second set and sequences that are at least partially complementary thereto. In the embodiment illustrated in FIG. 2A, the sequences of the probes of the second set are at least partially identical to the sequences of the probes of the first set. Each probe of the second set that is identical to a probe of the first set, i.e. 20a, 20b, and 20c, can bind nucleic acid 2 and strip the signal associated with labeled probes 10a, 10b, and 10c. Stripping the signals associated with signal probes 10a, 10b, and 10c, allows an additional set of labeled signal probes to be used to analyze nucleic acid 2.

Thus, as illustrated in FIG. 2A, nucleic acid 2 can then be contacted with a third set of probes comprising labeled signal probe subsets s5, s6, s7, and s8 under conditions effective to permit sequence-specific binding between labeled signal probes 10e, 10f, 10g, and 10h and sequences present on nucleic acid 2 that are at least partially complementary thereto. An optional wash step can be used to remove any unbound label probes, i.e. label probe 10f and 10g, prior to determining whether any labeled signal probes, i.e. label probes 10e and 10h, bound nucleic acid 2.

FIG. 2D illustrates the use of a labeled probe set comprising subsets, in which one or more of the subsets comprises one or more probes. In the embodiment illustrated in FIG. 2D, nucleic acid sample 2 is contacted with a first set of labeled signal probes comprising subsets s1, s2, s3, and s4. Subset s1 comprises probe 10a, subset s2 comprises probes 10b1 and 10b2, subset s3 comprises probe 10c and subset s4 comprises probes 10d1, 10d2, 10d3, and 10d4. The labeled probes are contacted with nucleic acid 2 under conditions effective to permit sequence-specific binding between probes 10a, 10b1, 10b2, and 10d4 and sequences present on nucleic acid 2 that are at least partially complementary thereto. Each subset bears a different distinguishable fluorophore label, i.e., subset s1 has label R, subset s2 has label Y, subset s3 has label B, and subset s4 has label G.

In addition to the embodiments illustrated in FIG. 2A and FIG. 2D, the unlabeled competitor probe set can be designed such that all of the probes have sequences that are at least partially complementary to the labeled signal probes, i.e. the embodiment illustrated in FIG. 2B, as well as the embodiment illustrated in FIG. 2E, in which some of the probes are designed to include sequences that are at least partially complementary to the labeled signal probes and some of the probes are designed to include sequences that are at least partially identical to the labeled signal probes.

Competitor probe sets can be added after the probe/target sequence complex has been formed and the signals detected, or competitor probes and signal probe sets can be added together. For example, FIG. 2A illustrates an embodiment in which the competitor probe set is added after the first signal probe/target sequence complex has been formed. Similarly, the third signal probe set is added after the competitor probe/target sequence complex has been formed. FIG. 2C illustrates an embodiment in which the competitor probe set and third signal probe set are added together. In the embodiment illustrated in FIG. 2C, the second set of competitor probes, i.e. competitor probes 20a, 20b, 20c, and 20d, is added after the first set of signal probes, i.e. 10a, 10b, 10c, and 10d. However, both the second set of competitor probes and the third set of signal probes, i.e. 10e, 10f, 10g, and 10h, are added together.

In embodiments in which a double stranded region of a nucleic acid comprises the target sequence, the labeled signal probes can be designed to have sequences that are at least partially identical and at least partially complementary to the region of the double stranded nucleic acid comprising the target sequence.

Once a nucleic acid or chromosome sample has been contacted with a labeled probe set, the detectable signals produced by the labels of the labeled probe set is monitored to determine which probes of the labeled probe set bound the nucleic acid. The detection system used can depend upon the nature of the detectable signal produced by the signal probe label, and will be apparent to those of skill in the art. Devices for measuring emissions from fluorescent signal labels (at one or more wavelengths) are available commercially. For example, digital images of interphase and metaphase cells after FISH can be acquired with a SensiCam 12-bit CCD Camera (PCO, Kelheim, Germany) attached to an Axioplan 2 microscope (Zeiss, Jena, Germany) using Northern Eclipse software (Empix Imaging, Mississauga, Ontario, Canada). As will be recognized by the skilled artisan, the filter sets used to equip the microscope depend upon the labels used in the assay. Images of each label can be acquired, and stored for further analysis.

Images of nucleic acid arrays can be acquired using a laser based array scanner with a CCD Camera.

Regardless of the detection system used, information regarding the location, identification and quantitation of the nucleic acid, chromosome, or organism should be provided by the system.

Kits for Detecting Target Sequences

The probe sets employed in the methods described herein can be packaged into kits. In some embodiments, the kits can be used for detecting chromosome abnormalities in human cells. In some other embodiments, the kits can be used for detecting target sequences associated with infectious disease, genetic disorders, or cellular disorders, and, accordingly, for diagnosing such maladies.

Such diagnostic kits may include, for example, a first set of labeled signal probes having sequences at least partially complementary to a region of a chromosome of interest, a second set of unlabelled competitor probes having sequences that are at least partially complementary and/or partially identical to the sequences of the probes of the first probe set, and a third set of labeled signal probes having sequences that are at least partially complementary to the same region or a different region of a chromosome of interest. The kit can also contain other suitably packaged reagents and materials as needed for the analysis of chromosome abnormalities, for example, cell culture medium, and buffers (i.e. for washing cells, for fixing cells, for hybridization, and counterstaining cells).

In other embodiments, the kits may include a first set and a third set of labeled probes having sequences that are at least partially complementary to a target sequence associated with a genetic disorder, cancer, or a pathogen, and a set of competitor probes having sequences that are at least partially complementary and/or partially identical to the sequences of the probes of the first probe set. In these embodiments, the reagents will be adjusted and modified as needed to allow for the analysis of the target sequence or sequences of interest.

Diagnostic Applications of the Present Methods and Compositions

In some embodiments, the methods and kits can be used to detect target sequences associated with chromosome abnormalities, genetic disorders, cellular disorders, or pathogens associated with infectious disease states. For example, the methods and kits can be used to detect chromosome abnormalities, i.e. numerical, present in preimplantation embryos. The preimplantation embryos can be obtained from humans, although embryos obtained from other organisms can also be analyzed (i.e., sheep, goats, pigs, cows, horses, dogs, cats, etc.).

In some embodiments, the methods and kits can be used to detect chromosome abnormalities present in preimplantation embryos. Abnormalities can be detected in metaphase or interphase chromosomes. For example, chromosome abnormalities associated with chromosomes X, Y, and 1-22 can be detected using FISH. Methods for obtaining and fixing blastomeres to glass slides from preimplantation embryos for analysis using FISH are well known in the art (see, e.g., Munne et al., 1998, Molecular Human Reproduction, 4(9):863-870; Bahce et al., 2000, Molecular Human Reproduction, 6(9):849-854; Bahce et al., 1999, Journal of Assisted Reproduction and Genetics, 16(4):176-181; Clouston et al., 1997, Hum Genet, 101:30-36; Delhanty et al., 1997, Hum Genet, 99:755-760; Harper et al., 1995, Prenatal Diagnosis, 15:41-49; Laverge, et al., 1997, Human Reproduction, 12(4):809-814; Munne et al., 1993, Human Reproduction, 8(12):2185-2191; Munne et al., 1998, Prenatal Diagnosis, 18:1459-1466; Plachot, et al., 1987, Human Reproduction, 2(1):29-35; Papadopoulos, et al., 1989, Human Reproduction, 4(1):91-98; Iwarsson et al., 1999, Hum Genet, 104:376-382; Angell, et al., 1986, Hum Genet, 72:333-339; Munne and Cohen, 1998, Human Reproduction Update, 4(6):842-855; Taneja et al., 2001, Genes, Chromosomes, & Cancer, 30:57-63; and Warbuton et al., 1986, In “Prenatal Genetics: Diagnosis and Treatment”, Academic Press, pp 23-40; all of which are incorporated herein by reference in their entireties). Methods for acquiring digital images of immobilized cells after FISH are also well known in the art, see, e.g., Taneja et al., 2001, Genes, Chromosomes, & Cancer, 30:57-63.

Chromosome specific sequences for chromosomes X, Y and 1-22 are also known to those of skill in the art (see, e.g., Munne et al., 1998, Molecular Human Reproduction, 4(9):863-870; Bahce et al., 2000, Molecular Human Reproduction, 6(9):849-854; Bahce et al., 1999, Journal of Assisted Reproduction and Genetics, 16(4):176-181; Clouston et al., 1997, Hum Genet, 101:30-36; Delhanty et al., 1997, Hum Genet, 99:755-760; Harper et al., 1995, Prenatal Diagnosis, 15:41-49; Laverge, et al., 1997, Human Reproduction, 12(4):809-814; Munne et al., 1993, Human Reproduction, 8(12):2185-2191; Munne et al., 1998, Prenatal Diagnosis, 18:1459-1466; Plachot, et al., 1987, Human Reproduction, 2(1):29-35; Papadopoulos, et al., 1989, Human Reproduction, 4(1):91-98; Iwarsson et al., 1999, Hum Genet, 104:376-382; Angell, et al., 1986, Hum Genet, 72:333-339; Munne and Cohen, 1998, Human Reproduction Update, 4(6):842-855; Taneja et al., 2001, Genes, Chromosomes, & Cancer, 30:57-63; and Warbuton et al., 1986, In “Prenatal Genetics: Diagnosis and Treatment”, Academic Press, pp 23-40; all of which are incorporated herein by reference in their entireties).

In some embodiments, non-nucleic acid probes for human chromosomes X, Y, 1, 2, 3, 6, 8, 10, 11, 12, 16, 17, and 18 can be used (see, e.g., U.S. patent application Ser. No. 09/520,760; the disclosure of which is incorporated herein by reference in its entirety), or human chromosomes X, Y, 1, 2, 3, 4, 6, 7, 8, 9, 10, 11, 12, 16, 17, 18 and 20 can be used (see, e.g., U.S. patent application Ser. No. 09/627,796; the disclosure of which is incorporated herein by reference in its entirety).

In some embodiments, the methods and kits can be used for the determination of pathogenic species in air, food, beverages, water, pharmaceutical products, personal care products, dairy products, environmental samples, mail and/or packaging, as well as in equipment used to process store and/or handle any of the foregoing. Additionally, the methods and kits can be used for the determination of pathogenic species in clinical samples and/or clinical environments. By way of a non-limiting example, the methods and kits can be used for the analysis of culture samples, or subcultures thereof. Other non-limiting examples of clinical samples include, but are not limited to: sputum, laryngeal swabs, gastric lavage, bronchial washings, biopsies, aspirates, expectorates, body fluids (e.g. spinal, pleural, pericardial, synovial, blood, pus, amniotic, and urine), bone marrow and/or tissue sections, or cultures or subcultures thereof. The methods and kits can also be used for the analysis of clinical specimens, equipment, fixtures and/or products used to treat humans and/or animals.

Bacterial, fungal, yeast, protozoan, and viral pathogens can be identified using the methods and kits described herein. In some embodiments, sets of labeled probes can be designed to identify pathogens in different genera. For example, one set of labeled probes can be designed to recognize yeast pathogens, another set of labeled probes can be designed to recognize fungal pathogens, and another set of labeled probes designed to recognize bacterial pathogens. As will be appreciated by a person of skill in the art, other combinations are also possible.

In other embodiments, pathogens can be determined at the species or genus level. For example, sets of labeled probes can be designed for bacterial pathogens belonging to different genera. Similarly, sets of labeled probes can be designed for different species within a genus of interest. Sets of labeled probes also can be designed to hybridize to different genera and species, i.e., the labeled probe set can comprise probes for different genera and different species that are in the same or different genera.

The probe sets described herein can be used to analyze biochips comprising a substrate with an array of target sequences. As will be appreciated by a person of skill in the art, the size of the array will depend on the composition and end use of the array. For example, a plurality of nucleic acids comprising sequences of interest can be “arrayed” on a biochip as described in U.S. Pat. No. 6,110,676 (the disclosure of which is incorporated herein by reference in its entirety). These biochips can then be used in a wide variety of ways, including but not limited to, diagnosis (e.g., detecting the presence of specific target sequences), screening (e.g., determining if a pathogen of interest is present in a sample), and single-nucleotide polymorphism (SNP) analysis (e.g., detecting the presence or absence of SNPs associated with certain disease states or metabolic disorders).

All publications, patent applications, and similar materials mentioned herein are hereby incorporated by reference in their entirety for any purpose. In the event that one or more of the incorporated materials differs from or contradicts this application, including but not limited to defined terms, term usage, described techniques, or the like, this application controls.

The following Examples are illustrative of the disclosed compositions and methods, and are not intended to limit the scope of the various embodiments described herein. Without departing from the spirit and scope of the present teachings, various changes and modifications of the present teachings will be clear to one skilled in the art and can be made to adapt the present teachings to various uses and conditions. Thus, other embodiments are encompassed.

### Examples

Aspects of the present teachings may be further understood in light of the following examples, which should not be construed as limiting the scope of the present teachings in any way.

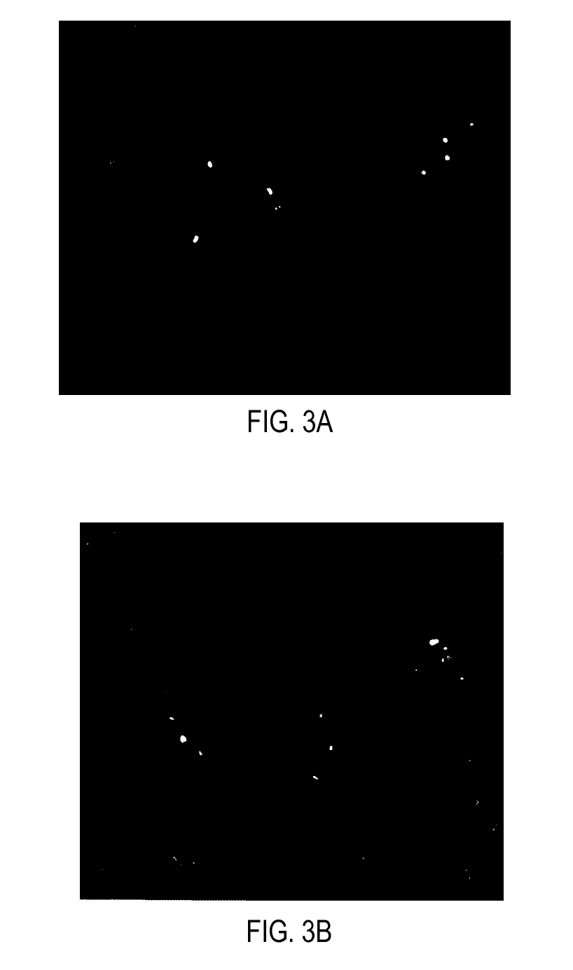

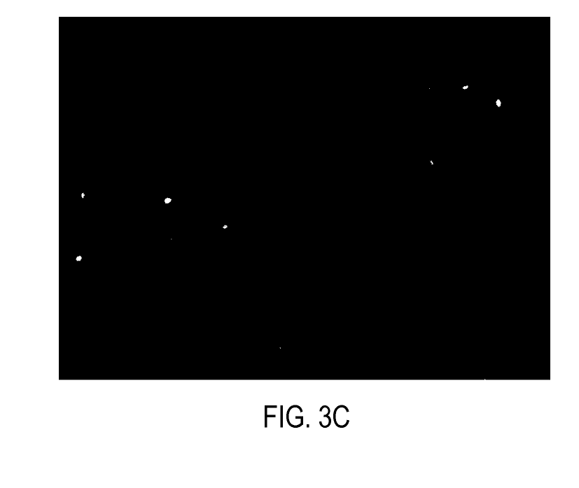

The attached images (i.e. FIG. 3A, 3B, 3C) were from an experiment to demonstrate proof of principle. Three panels of probes were utilized: Panel 1—13/21 combined probe set labeled with fluorescein; Panel 2—probe sets for chromosomes 1, 16, 17 labeled with Diethylaminocoumarin (DEAC), Cy3 and fluorescein respectively plus the unlabeled competitor probes for chromosome 13/21 in panel 1; Panel 3—probe sets for chromosomes 18, X, Y labeled with Diethylaminocoumarin (DEAC), Cy3 and fluorescein respectively plus the unlabeled competitor probes for chromosomes 1, 16 and 17 in panel 2. The first panel was allowed to hybridize to metaphase and interphase cells on a slide using the PNA FISH protocol described by Taneja et al (Genes Chromosomes Cancer. 2001 January; 30(1):57-63). For the second and subsequent panels the procedure of Taneja et al was modified slightly by reducing the 5 minutes denaturation step at the start of the protocol to 1 minute. All images were captured using a Photometrics Coolsnap FX CCD camera attached to an Olympus AX70 microscope and were pseudo-colored using Open Lab software (Improvision).

In FIG. 3A, the panel 1 labeled signal probe set was hybridized to a metaphase cell, with the green fluorescent signals indicating that the probes have hybridized to the centromeres of chromosomes 13 and 21. DAPI was used for counterstaining to provide the location of chromosomes in the metaphase cell.

In the FIG. 3B image, the same metaphase cell was then hybridized with the panel 2 probe sets. Two probes sets were using in panel 2: 1) a labeled signal probe set for chromosomes 1, 16, 17, wherein the probes were labeled with Diethylaminocoumarin (DEAC), Cy3 and fluorescein; and, 2) an unlabeled competitor probe set for chromosome 13/21 in panel 1. In the FIG. 3B image, the centromeres of chromosomes 1, 16, 17 fluoresce blue (computer enhanced to appear white), red and green respectively. The fluorescent signal from the centromeres of chromosomes 1.3 and 21 has been stripped by using unlabelled competitor probes substantially identical to the labeled signal probes in panel 1.

The FIG. 3C image is the same metaphase cell used in the two previous steps, but hybridized with the panel 3 probe sets. The panel 3 probe sets comprise: 1) a labeled signal probe set for chromosomes 18, X, Y, wherein the probes are labeled with Diethylaminocoumarin (DEAC), Cy3 and fluorescein; and 2) the unlabeled competitor probes for chromosomes 1, 16 and 17 in panel 2. In the FIG. 3C image, the centromeres of chromosomes X and 18 fluoresce blue (computer enhanced to appear white) and red respectively. The Y chromosome is not present in the cell as the metaphase cell is from a female cell line. The fluorescent signals from chromosomes 1, 16 and 17 in the previous panel have been stripped by the unlabelled competitor probe versions of these probes in the panel 3 probe set.

1-26. (canceled)

27. A method for use in diagnosing the presence or absence of chromosomal anueploidy, comprising the steps of:
(a) contacting chromosomes of an immobilized cell with a first set of probes under conditions effective to permit sequence-specific binding of the probes of the first set and sequences that are least partially complementary thereto, wherein the first set of probes comprises n subsets of u probes, wherein n is an integer from 1 to 12, and each u, independently of the others, is an integer from 1 to 20, the sequences of the probes of each of the n subsets are specific for a chromosome of interest, a different chromosome per set, and the u probes of each subset are labeled with the same label but each n subset comprises a different, spectrally resolvable label;
(b) optionally removing unbound probes of the first set;
(c) determining which probes of the first set bound their respective chromosomes of interest;
(d) contacting the chromosomes with a second set of probes under conditions effective to permit sequence specific binding between probes of the second set and sequences that are at least partially complementary thereto, wherein the sequences of the probes of the second set are at least partially identical and/or partially complementary to the sequences of the probes of the first set;
(e) contacting the chromosomes with a third set of probes of m sets of v probes under conditions effective to permit sequence specific binding between probes of the third set and sequences that are at least partially complementary thereto, wherein m is an integer from 1 to 12, and each v, independently of the others, is an integer from 1 to 20, the sequences of the probes of each of the m subsets are specific for a chromosome of interest, a different chromosome per set, and the v probes of each subset are labeled with the same label but each m subset comprises a different, spectrally resolvable label;
(f) optionally removing unbound probes of the third set;
(g) determining which probes of the third set bound their respective chromosomes of interest;
(h) optionally repeating steps (d) through (g) from 1 to 4 times, wherein the sequences of the probes corresponding to step (d) are substantially identical and/or substantially complementary to the sequences of the probes of the immediately preceding set of probes; and
(i) diagnosing therefrom the presence or absence of anueploidy in the chromosomes of the cell.

28. A method for use in diagnosing the presence or absence of chromosomal anueploidy, comprising the steps of:
(a) contacting the chromosomes of an immobilized cell with one or more sets of labeled probes specific for each of a plurality of different chromosomes of interest, wherein the labels of probes specific for different chromosomes are distinguishable from one another; wherein said contacting is carried out under conditions effective to permit sequence specific binding between labeled probes and sequences that are at least partially complementary thereto,
(b) optionally removing unbound labeled probes;
(c) determining which of the labeled probes bound their respective chromosomes of interest;
(d) contacting the chromosomes with one or more sets of unlabeled probes, wherein the sequences of the of the unlabeled probes are at least partially identical and/or partially complementary to the sequences of the labeled probes;
(e) contacting the chromosomes with one or more sets of labeled probes specific for each of a plurality of different chromosomes of interest, wherein the sets of labeled probes are different from those of step (a), the labels of probes specific for different chromosomes are distinguishable from one another; and said contacting is carried out under conditions effective to permit sequence specific binding between labeled probes and sequences that are at least partially complementary thereto,
(f) optionally removing unbound labeled probes;
(g) determining which labeled probes bound their respective chromosomes of interest;
(h) optionally repeating steps (d) through (g) from 1 to 4 times, wherein the sequences of the probes corresponding to step (d) are substantially identical and/or substantially complementary to the sequences of the probes of the immediately preceding set of probes; and
(i) diagnosing therefrom the presence or absence of aneuploidy in the chromosomes of the cell.

29. (canceled)

30. A kit for use in analyzing chromosomal aneuploidy, comprising:
a first set of n subsets of u probes having sequences that are at least complementary to a region of a chromosome of interest, wherein n is an integer from 1 to 7, and each u is, independently from the others, an integer from 1 to 20, and each u probe of a subset is labeled with the same label but each of the different n subsets is labeled with a different, distinguishable detectable label;
a second set of probes having sequences that are at least partially complementary and/or partially identical to the sequences of the probes of the first set, wherein the number of probes in the second set comprises at least the sum of the number of probes in the first set; and,
a third set of m subsets of v probes having sequences that are at least partially complementary to a region of a chromosome of interest, wherein m is an integer from 1 to 7, and each v is, independently from the others, an integer from 1 to 20, and each v probe of a subset is labeled with the same label but each of the different m subsets is labeled with a different, distinguishable detectable label.

31. The method of claim 27, in which each of the n subsets comprises a single probe, and/or each of the m subsets comprises a single probe.

32. The method of claim 27, in which one or more of the n subsets comprises a single probe, and/or one or more of the m subsets comprises a single probe.

33. The method of claim 27, in which the probes of the second set are unlabeled.

34. The method of claim 27, in which each of the n subsets comprises a plurality of from 2 to 12 probes, each of which has a different sequence, and/or each of the m subsets comprises a plurality of from 2 to 12 probes, each of which has a different sequence.

35. The method of claim 34, in which the probes of the n and m subsets are chromosome-specific.

36. The method of claim 35, in which the probes of each of the n and m subsets are specific for a different chromosome.

37. The method of claim 27, in which the sequences of the probes of the second set are identical to the sequences of the probes of the first set.

38. The method of claim 27, in which the sequences of the probes of the second set are completely complementary to the sequences of the probes of the first set.

39. The method of claim 27, in which the sequences of some of the probes of the second set are identical to the sequences of some of the probes of the first set and the sequences of the remainder of the probes of the second set are completely complementary to the sequences of the remainder of the probes of the first set.

40. The method of claim 27, in which steps (d) and (e) are carried out simultaneously.

41. The method of claim 27, in which the labels of the first and third sets of probes are fluorescent labels.

42. The method of claim 41, in which the fluorescent labels are selected from a set of spectrally resolvable fluorophores.

43. The method of claim 27, in which n and m are each, independently of one another, integers selected from 1 to 4.

44. The method of claim 27, in which n and m are each the same integer selected from 1 to 4.

45. The method of claim 27, in which the probes of the first, second and/or third sets are PNA probes.

46. The method of claim 27, in which the probes of the first, second and/or third sets are LNA probes.

47. The method of claim 27, in which the probes of the first, second and/or third sets are PNA/LNA chimeras.

48. The method of claim 27, further comprising repeating steps (d) through (g) from 1 to 8 times with subsequent sets of probes, wherein the sequences of the probes corresponding to step (d) are at least partially identical and/or partially complementary to the sequences of the probes of the immediately preceding set of probes.

49. The method of claim 48, comprising repeating steps (d) through (g) from 1 to 4 times.

50. The method of claim 48, in which the sequences of the probes of the sets corresponding to step (d) are identical to the sequences of the probes of the immediately preceding set of probes.

51. The method of claim 48, in which the sequences of the probes of the sets corresponding to step (d) are completely complementary to the sequences of the probes of the immediately preceding set of probes.

52. The method of claim 48, in which the sequences of some of the probes of the sets corresponding to step (d) are identical to the sequences of probes of the immediately preceding set of probes and the sequences of the remainder of the probes of the sets corresponding to step (d) are completely complementary to the sequences of the remainder of the probes of the immediately preceding set of probes.

53. The method of claim 48, in which steps (d) and (e) are carried out simultaneously.

54. The method of claim 28, in which the labels of the one or more sets of labeled probes are fluorescent labels.

55. The method of claim 54, in which the fluorescent labels are selected from a set of spectrally resolvable fluorophores.

56. The method of claim 28, in which the probes of the one or more sets of labeled probes are PNA probes.

57. The method of claim 28, in which the probes of the one or more sets of labeled probes are LNA probes.

58. The method of claim 28, in which the probes of the one or more sets of labeled probes are PNA/LNA chimeras.

59. The method of claim 28, further comprising repeating steps (d) through (g) from 1 to 8 times with subsequent sets of probes, wherein the sequences of the probes corresponding to step (d) are at least partially identical and/or partially complementary to the sequences of the probes of the immediately preceding set of probes.

60. The method of claim 59, comprising repeating steps (d) through (g) from 1 to 4 times.

61. The method of claim 59, in which the sequences of the probes of the sets corresponding to step (d) are identical to the sequences of the probes of the immediately preceding set of probes.

62. The method of claim 59, in which the sequences of the probes of the sets corresponding to step (d) are completely complementary to the sequences of the probes of the immediately preceding set of probes.

63. The method of claim 59, in which the sequences of some of the probes of the sets corresponding to step (d) are identical to the sequences of probes of the immediately preceding set of probes and the sequences of the remainder of the probes of the sets corresponding to step (d) are completely complementary to the sequences of the remainder of the probes of the immediately preceding set of probes.

64. The method of claim 59, in which steps (d) and (e) are carried out simultaneously.

65. The kit of claim 30, in which each of the n subsets comprises a single probe, and/or each of the m subsets comprises a single probe.

66. The kit of claim 30, in which one or more of the n subsets comprises a single probe, and/or one or more of the m subsets comprises a single probe.

67. The kit of claim 30, in which the probes of the second set are unlabeled.

68. The kit of claim 30, in which each of the n subsets comprises a plurality of from 2 to 12 probes, each of which has a different sequence, and/or each of the m subsets comprises a plurality of from 2 to 12 probes, each of which has a different sequence.

69. The kit of claim 68, in which the probes of the n and m subsets are chromosome-specific.

70. The kit of claim 69, in which the probes of each of the n and m subsets are specific for a different chromosome.

71. The kit of claim 30, in which the sequences of the probes of the second set are identical to the sequences of the probes of the first set.

72. The kit of claim 30, in which the sequences of the probes of the second set are completely complementary to the sequences of the probes of the first set.

73. The kit of claim 30, in which the sequences of some of the probes of the second set are identical to the sequences of some of the probes of the first set and the sequences of the remainder of the probes of the second set are completely complementary to the sequences of the remainder of the probes of the first set.

74. The kit of claim 30, in which the labels of the first and third sets of probes are fluorescent labels.

75. The kit of claim 74, in which the fluorescent labels are selected from a set of spectrally resolvable fluorophores.

76. The kit of claim 30, in which n and m are each, independently of one another, integers selected from 1 to 4.

77. The kit of claim 30, in which n and m are each the same integer selected from 1 to 4.

78. The kit of claim 30, in which the probes of the first, second and/or third sets are PNA probes.

79. The kit of claim 30, in which the probes of the first, second and/or third sets are LNA probes.

80. The kit of claim 30, in which the probes of the first, second and/or third sets are PNA/LNA chimeras.
